SEQUENCE LISTING (40 sequences)

SEQ ID NO: 1 - DNA, 21 bp, Artificial Sequence
  Oligonucleotide designed to act as PCR primer for detection of preproglucagon (alpha cell specific marker) gene
atttactttgtggctggattg

SEQ ID NO: 2 - DNA, 21 bp, Artificial Sequence
  Oligonucleotide designed to act as PCR primer for detection of preproglucagon (alpha cell specific marker) gene
tgtcagtgatcttggtttgaa

SEQ ID NO: 3 - DNA, 20 bp, Artificial Sequence
  Oligonucleotide designed to act as PCR primer for detection of preproinsulin I (beta cell specific marker) gene
ctgttggtgcacttcctacc

SEQ ID NO: 4 - DNA, 20 bp, Artificial Sequence
  Oligonucleotide designed to act as PCR primer for detection of preproinsulin I (beta cell specific marker) gene
gcagtagttctccagctggt

SEQ ID NO: 5 - DNA, 21 bp, Artificial Sequence
  Oligonucleotide designed to act as PCR primer for detection of preproinsulin II (beta cell specific marker) gene
tcaagcagcacctttgtggtt

SEQ ID NO: 6 - DNA, 21 bp, Artificial Sequence
  Oligonucleotide designed to act as PCR primer for detection of preproinsulin II (beta cell specific marker) gene
gttgcagtagttctccagctg

SEQ ID NO: 7 - DNA, 20 bp, Artificial Sequence
  Oligonucleotide designed to act as PCR primer for detection of pancreatic polypetide (gamma cell specific marker) gene
ctccctgtttctcgtatcca

SEQ ID NO: 8 - DNA, 20 bp, Artificial Sequence
  Oligonucleotide designed to act as PCR primer for detection of pancreatic polypetide (gamma cell specific marker) gene
agagcagggaatcaagccaa

SEQ ID NO: 9 - DNA, 19 bp, Artificial Sequence
  Oligonucleotide designed to act as PCR primer for detection of preprosomatostatin (delta cell specific marker) gene
ctctgcatcgtcctggctt

SEQ ID NO: 10 - DNA, 21 bp, Artificial Sequence
  Oligonucleotide designed to act as PCR primer for detection of preprosomatostatin (delta cell specific marker) gene
caggatgtgaatgtcttccag

SEQ ID NO: 11 - DNA, 20 bp, Artificial Sequence
  Oligonucleotide designed to act as PCR primer for detection of amylase 2 (exocrine cell specific marker) gene
agtacctgtggaagttacct

SEQ ID NO: 12 - DNA, 20 bp, Artificial Sequence
  Oligonucleotide designed to act as PCR primer for detection of amylase 2 (exocrine cell specific marker) gene
acacaagggctctgtcagaa

SEQ ID NO: 13 - DNA, 20 bp, Artificial Sequence
  Oligonucleotide designed to act as PCR primer for detection of hormone-sensitive lipase (exocrine cell specific marker) gene
tcttcttcgagggtgatgaa

SEQ ID NO: 14 - DNA, 20 bp, Artificial Sequence
  Oligonucleotide designed to act as PCR primer for detection of hormone-sensitive lipase (exocrine cell specific marker) gene
taccttgctgtcctgtcctt

SEQ ID NO: 15 - DNA, 21 bp, Artificial Sequence
  Oligonucleotide designed to act as PCR primer for detection of ipf1/pdx1 gene
ttacaagctcgctgggatcac

SEQ ID NO: 16 - DNA, 21 bp, Artificial Sequence
  Oligonucleotide designed to act as PCR primer for detection of ipf1/pdx1 gene
aggtcaccgcacaatcttgct

SEQ ID NO: 17 - DNA, 20 bp, Artificial Sequence
  Oligonucleotide designed to act as PCR primer for detection of c-met gene
agccagtaatgatctcaatg

SEQ ID NO: 18 - DNA, 18 bp, Artificial Sequence
  Oligonucleotide designed to act as PCR primer for detection of c-met gene
tcaggataggggacaggt

SEQ ID NO: 19 - DNA, 21 bp, Artificial Sequence
  Oligonucleotide designed to act as PCR primer for detection of hypoxanthine phosphoribosyltransferase (HPRT, positive control) gene
ctgtaatgatcagtcaacggc

SEQ ID NO: 20 - DNA, 21 bp, Artificial Sequence
  Oligonucleotide designed to act as PCR primer for detection of hypoxanthine phosphoribosyltransferase (HPRT, positive control) gene
ggcctataggctcatagtgca

SEQ ID NO: 21 - DNA, 21 bp, Artificial Sequence
  Oligonucleotide designed to act as PCR primer for detection of albumin (hepatocyte/biliary cell marker) gene
catgacaccatgcctgctgat

SEQ ID NO: 22 - DNA, 22 bp, Artificial Sequence
  Oligonucleotide designed to act as PCR primer for detection of albumin (hepatocyte/biliary cell marker) gene
ctctgatcttcaggaagtgtac

SEQ ID NO: 23 - DNA, 21 bp, Artificial Sequence
  Oligonucleotide designed to act as PCR primer for detection of alpha-fetoprotein (hepatocyte/biliary cell marker) gene
actcaccccaaccttcctgtc

SEQ ID NO: 24 - DNA, 21 bp, Artificial Sequence
  Oligonucleotide designed to act as PCR primer for detection of alpha-fetoprotein (hepatocyte/biliary cell marker) gene
cagcagtggctgataccagag

SEQ ID NO: 25 - DNA, 21 bp, Artificial Sequence
  Oligonucleotide designed to act as PCR primer for detection of glucose-6-phosphatase (hepatocyte/biliary cell marker) gene
aacccattgtgaggccagagg

SEQ ID NO: 26 - DNA, 21 bp, Artificial Sequence
  Oligonucleotide designed to act as PCR primer for detection of glucose-6-phosphatase (hepatocyte/biliary cell marker) gene
tactcattacactagttggtc

SEQ ID NO: 27 - DNA, 21 bp, Artificial Sequence
  Oligonucleotide designed to act as PCR primer for detection of transthyretin (hepatocyte/biliary cell marker) gene
tggtatttgtgtctgaagctg

SEQ ID NO: 28 - DNA, 21 bp, Artificial Sequence
  Oligonucleotide designed to act as PCR primer for detection of transthyretin (hepatocyte/biliary cell marker) gene
ttaataagaatgcttcacggc

SEQ ID NO: 29 - DNA, 20 bp, Artificial Sequence
  Oligonucleotide designed to act as PCR primer for detection of cytokeratin 19 (hepatocyte/biliary cell marker) gene
gtcctacagattgacaatgc

SEQ ID NO: 30 - DNA, 21 bp, Artificial Sequence
  Oligonucleotide designed to act as PCR primer for detection of cytokeratin 19 (hepatocyte/biliary cell marker) gene
cacgctctggatctgtgacag

SEQ ID NO: 31 - DNA, 21 bp, Artificial Sequence
  Oligonucleotide designed to act as PCR primer for detection of cytokeratin 18 (hepatocyte/biliary cell marker) gene
ggacctcagcaagatcatggc

SEQ ID NO: 32 - DNA, 21 bp, Artificial Sequence
  Oligonucleotide designed to act as PCR primer for detection of cytokeratin 18 (hepatocyte/biliary cell marker) gene
ccacgatcttacgggtagttg

SEQ ID NO: 33 - DNA, 20 bp, Artificial Sequence
  Oligonucleotide designed to act as PCR primer for detection of cytokeratin 8 (hepatocyte/biliary cell marker) gene
agtctcagatctcagacacg

SEQ ID NO: 34 - DNA, 20 bp, Artificial Sequence
  Oligonucleotide designed to act as PCR primer for detection of cytokeratin 8 (hepatocyte/biliary cell marker) gene
ccataggatgaactcagtcc

SEQ ID NO: 35 - DNA, 21 bp, Artificial Sequence
  Oligonucleotide designed to act as PCR primer for detection of biliary glycoprotein (hepatocyte/biliary cell marker) gene
gaactagactctgtcaccctg

SEQ ID NO: 36 - DNA, 20 bp, Artificial Sequence
  Oligonucleotide designed to act as PCR primer for detection of biliary glycoprotein (hepatocyte/biliary cell marker) gene
gccagacttcctggaataga

SEQ ID NO: 37 - DNA, 21 bp, Artificial Sequence
  Oligonucleotide designed to act as PCR primer for detection of intestinal fatty acid binding protein (fabp-2; gastric cell/enterocyte marker) gene
attcgacggcacgtggaaagt

SEQ ID NO: 38 - DNA, 21 bp, Artificial Sequence
  Oligonucleotide designed to act as PCR primer for detection of intestinal fatty acid binding protein (fabp-2; gastric cell/enterocyte marker) gene
aagaatcgcttggcctcaact

SEQ ID NO: 39 - DNA, 21 bp, Artificial Sequence
  Oligonucleotide designed to act as PCR primer for detection of pepsinogen F (gastric cell/enterocyte marker) gene
acctagacctggtctacattg

SEQ ID NO: 40 - DNA, 21 bp, Artificial Sequence
  Oligonucleotide designed to act as PCR primer for detection of pepsinogen F (gastric cell/enterocyte marker) gene
agtgaagctctccatggtagt

(19) United States
(12) Patent Application Publication
(10) Pub. No.: US 2005/0003529 A1
(43) Pub. Date: Jan. 6, 2005

(54) Stem cells and method of separating the same

(76) Inventors: Hideki Taniguchi, Ushiku-shi (JP); Atsushi Suzuki, Kiryu-shi (JP)

Correspondence Address: NATH & ASSOCIATES, 1030 15th STREET, NW, 6TH FLOOR, WASHINGTON, DC, 20005, US

(21) Appl. No.: 10/693,712

(22) Filed: Oct. 27, 2003

Related U.S. Application Data
(63) Continuation of application No. PCT/JP02/04084, filed on Apr. 24, 2002.

(30) Foreign Application Priority Data
Apr. 24, 2001 (JP) 126315/2001

(51) Int. Cl. C12Q 1/68; C12N 5/08; G01N 33/53; G01N 33/567

(52) U.S. Cl. 435/366; 435/6; 435/7.2

(57) ABSTRACT

The present invention provides a method of separating a pancreatic stem cell from the pancreas of a mammal, a method of identifying a pancreatic stem cell of a mammal, and use of a pancreatic stem cell that can be separated or identified by this method. More particularly, the present invention provides a method of separating or identifying a pancreatic stem cell of a mammal, which includes analyzing the state of expression of 2 or more marker proteins selected from the group consisting of c-Met, c-Kit, CD45, TER119 and Flk-1, or a gene encoding the marker protein.

### TECHNICAL FIELD

The present invention relates to a pancreatic stem cell of a mammal and a separation or identification method thereof, as well as use of the cell.

### BACKGROUND ART

A method of separating a hepatic progenitor cell from the liver of a fetal mouse using [c-Kit⁻, CD49f⁺, CD29⁺, CD45⁻, TER119⁻] as a marker (Hepatology 32(6), 1230-1239 (2000)) and a method of separating a hepatic stem cell using [c-Met⁺, CD49f⁺/low, c-Kit⁻, CD45⁻, TER119⁻] as a marker (Hideki Taniguchi: Records of the 117th Japanese Association of Medical Sciences Symposium, Stem cell and cell therapy, 80-89 (2001)) have been reported. It has been considered that an organ has a unique stem cell thereof, but the presence of a marker common to the stem cells has not been expected. In particular, a method of separation or identification of a pancreatic stem cell that differentiate variously and exerts wide-ranging physiological functions has not been established at present.

### DISCLOSURE OF THE INVENTION

It is an object of the present invention to provide a method of separating or identifying a pancreatic stem cell. Another object of the present invention is to provide a pancreatic stem cell and to provide the stem cell as a pharmaceutical agent and/or a reagent.

The present inventors have conducted intensive studies in an attempt to achieve the above-mentioned object and found an expression pattern of a marker of [c-Met⁺, c-Kit⁻, CD45⁻, TER119⁻] common to pancreatic stem cells, and succeeded in preferably separating and identifying a pancreatic stem cell utilizing the characteristics. Further detailed studies have resulted in the finding that pancreatic stem cells can be preferably further separated and identified, using the expression pattern of a marker [c-Met⁺, c-Kit⁻, CD45⁻, TER119⁻, Flk-1⁻] as an index, which resulted in the completion of the present invention. Accordingly, the present invention provides the-following.

(1) A method of separating a pancreatic stem cell from the pancreas of a mammal using two or more kinds of substances having specific affinity for a marker protein selected from the group consisting of c-Met, c-Kit, CD45 and TER119 or a gene encoding the same.

(2) A method of separating a pancreatic stem cell from the pancreas of a mammal using two or more kinds of substances having specific affinity for a marker protein selected from the group consisting of c-Met, c-Kit, CD45, TER119 and Flk-1 or a gene encoding the same.

(3) The method of the above-mentioned (1) or (2), wherein the substance having specific affinity is an antibody against the marker protein.

(4) A method of identifying a pancreatic stem cell of a mammal using two or more kinds of substances having specific affinity for a marker protein selected from the group consisting of c-Met, c-Kit, CD45 and TER119 or a gene encoding the same.

(5) A method of identifying a pancreatic stem cell of a mammal using two or more kinds of substances having specific affinity for a marker protein selected from the group consisting of c-Met, c-Kit, CD45, TER119 and Flk-1 or a gene encoding the same.

(6) The method of the above-mentioned (4) or (5), wherein the substance having specific affinity is an antibody against the marker protein.

(7) A method of separating a pancreatic stem cell from the pancreas of a mammal, which comprises a step of analyzing the expression state of two or more marker proteins selected from the group consisting of c-Met, c-Kit, CD45 and TER119 or a gene encoding the same.

(8) A method of separating a pancreatic stem cell from the pancreas of a mammal, which comprises a step of analyzing the expression state of two or more marker proteins selected from the group consisting of c-Met, c-Kit, CD45, TER119 and Flk-1 or a gene encoding the same.

(9) A method of identifying a pancreatic stem cell from the pancreas of a mammal, which comprises a step of analyzing the expression state of two or more marker proteins selected from the group consisting of c-Met, c-Kit, CD45 and TER119 or a gene encoding the same.

(10) A method of identifying a pancreatic stem cell from the pancreas of a mammal, which comprises a step of analyzing the expression state of two or more marker proteins selected from the group consisting of c-Met, c-Kit, CD45, TER119 and Flk-1 or a gene encoding the same.

(11) A pancreatic stem cell that can be separated-from the pancreas of a mammal by the method described in any of the above-mentioned (1), (2), (7) and (8).

(12) The pancreatic stem cell of the above-mentioned (11), which shows four markers of c-Met, c-Kit, CD45 and TER119 in a pattern of c-Met⁺, c-Kit⁻, CD45⁻, TER119⁻.

(13) The pancreatic stem cell of the above-mentioned (11), which shows five markers of c-Met, c-Kit, CD45, TER119 and Flk-1 in a pattern of c-Met⁺, c-Kit⁻, CD45⁻, TER119⁻, Flk-1⁻.

(14) An agent for the prophylaxis or treatment of a pancreatic hypofunctional disease, which comprises the pancreatic stem cell of the above-mentioned (12) or (13), or a cell differentiated from the stem cell.

(15) An agent for the prophylaxis or treatment of a hypofunctional disease of the liver•bile duct or the stomach intestine, which comprises the pancreatic stem cell of the above-mentioned (12) or (13), or a cell differentiated from the stem cell.

(16) A method of screening a substance that induces differentiation of a pancreatic stem cell of a mammal, which comprises the following steps:
(i) a step of reacting a pancreatic stem cell with a test substance, and
(ii) a step of determining the expression of a pancreatic marker in the cell after the reaction.

(17) A method of screening a substance that induces differentiation into liver•bile duct or stomach•intestine of a mammal, which comprises the following steps:
(i) a step of reacting a pancreatic stem cell of the above-mentioned (12) or (13) with a test substance, and
(ii) a step of determining the expression of a liver•bile duct or stomach•intestine marker in the cell after the reaction.

(18) A method of screening a substance that regulates a pancreatic function of a mammal, which comprises the following steps:
(i) a step of reacting a pancreatic stem cell or a cell differentiated from the stem cell with a test substance, and
(ii) a step of determining the expression of a pancreatic marker in the cell after the reaction.

(19) A method of screening a substance that regulates the function of liver•bile duct or stomach•intestine of a mammal, which comprises the following steps:
(i) a step of reacting a pancreatic stem cell of the above-mentioned (12) or (13) or a cell differentiated from the stem cell with a test substance, and
(ii) a step of determining the expression of a liver•bile duct or stomach•intestine marker in the cell after the reaction.

(20) A cloned pluripotent pancreatic stem cell, which satisfies at least 2 of the characteristics selected from the group consisting of c-Met⁺, c-Kit⁻, CD45⁻ and TER119⁻.

(21) A cloned pluripotent pancreatic stem cell, which satisfies at least 2 of the characteristics selected from the group consisting of c-Met⁺, c-Kit⁻, CD45⁻, TER119⁻ and Flk-1⁻.

(22) A pharmaceutical composition, which comprises:
i) a cloned pluripotent pancreatic stem cell of the above-mentioned (20) or (21); and
ii) a pharmaceutically acceptable carrier.

(23) A purified composition, which comprises: a cloned pluripotent pancreatic stem cell of the above-mentioned (20) or (21).

(24) Tissue regenerated from a cloned pluripotent pancreatic stem cell of the above-mentioned (20) or (21).

(25) An organ regenerated from a cloned pluripotent pancreatic stem cell of the above-mentioned (20) or (21).

(26) A method of transplanting a cloned pluripotent pancreatic stem cell into a host, which comprises:
i) obtaining the cloned pluripotent pancreatic stem cell of the above-mentioned (20) or (21); and
ii) transplanting said stem cell into the host.

(27) A method of producing a pancreatic stem cell, which comprises:
i) providing cells from the pancreas of a mammal; and
ii) selecting cells which satisfies at least 2 of the marker protein expression patterns selected from the group consisting of c-Met⁺, c-Kit⁻, CD45⁻ and TER119⁻, or a gene encoding the same.

(28) A method of producing a pancreatic stem cell, which comprises:
i) providing cells from the pancreas of a mammal; and
ii) selecting cells which satisfies at least 2 of the marker protein expression patterns selected from the group consisting of c-Met⁻, c-Kit⁻, CD45⁻, TER119⁻ and Flk-1⁻ or a gene encoding the same.

(29) The method according to the above-mentioned (27) or (28), wherein the step of selecting cells further comprises selecting cells using an antibody having a specific affinity against the marker protein.

(30) A method of screening for a pancreatic stem cell of a mammal, which comprises;
i) containing a population of cells with an antibody having specific affinity for a marker protein selected from the group consisting of c-Met, c-Kit, CD45 and TER119, or a gene encoding the same; and
ii) fractionating the population of cells to obtain a cell having multi-differentiation ability.

(31) The method of the above-mentioned (30), wherein the cells are pancreatic cells.

(32) The method of the above-mentioned (30), wherein the cells express a marker protein pattern of c-Met⁺, c-Kit⁻, CD45⁻ and TER119⁻.

(33) The method of the above-mentioned (30), wherein the cells express a marker protein pattern of c-Met⁺, c-Kit⁻, CD45⁻, TER119⁻ and Flk-1⁻.

(34) An agent for the prophylaxis or treatment of a pancreatic hypofunctional disease, which comprises the pancreatic stem cell of the above-mentioned (12), (13), (20) or (21), or a cell differentiated from the stem cell.

(35) The agent of the above-mentioned (34), wherein the pancreatic hypofunctional disease is a disease selected from the group consisting of diabetes chronic pancreatitis, autoimmune pancreatitis and pancreatic functional disorder from surgical removal of all or part of a pancreas.

(36) An agent for the prophylaxis or treatment of a hypofunctional disease of the liver•bile duct, which comprises the pancreatic stem cell of the above-mentioned (12), (13), (20) or (21), or a cell differentiated from the stem cell.

(37) The agent of the above-mentioned (36), wherein the hypofunctional disease of the liver•bile duct is a disease selected from the group consisting of acute hepatitis, chronic hepatitis, metabolic liver disease and hepatic functional disorder from surgical removal of all or part of a liver.

(38) An agent for the prophylaxis or treatment of a hypofunctional disease of the stomach•intestine, which comprises the pancreatic stem cell of the above-mentioned (12), (13), (20) or (21), or a cell differentiated from the stem cell.

(39) The agent of the above-mentioned (38), wherein the hypofunctional disease of the stomach•intestine is a disease selected from the group consisting of short bowel syndrome, inflammatory bowel disease, and stomach functional disorder from surgical removal of all or part of a stomach.

(40) A pancreatic stem cell containing device, which comprises:
at least one cloned pluripotent pancreatic stem cell, a container which houses the at least one stem cell, a substrate within the housing for anchoring the cells, wherein the container is permeable and allows entry-of essential nutrients and discharge of pancreatic stem cell products.

(41) The devise of the above-mentioned (40), wherein the device is transplantable into a mammalian host.

(42) An antibody kit, which comprises:
i) a container; and
ii) one or more antibodies which have a specific affinity for c-Met, c-Kit, CD45 and TER119.

(43) An antibody kit, which comprises:
i) a container; and
ii) one or more antibodies which have a specific affinity for c-Met, c-Kit, CD45, TER119 and Flk-1.

### DETAILED DESCRIPTION OF THE INVENTION

The present invention provides a method of separating or identifying a pancreatic stem cell from the pancreas of a mammal, using a substance having specific affinity for each of 2 or more, preferably 3 or more, most preferably all of the 4, marker proteins selected from the group consisting of c-Met, c-Kit, CD45 and TER119, or a gene encoding same.

Furthermore, the present invention provides a method of separating or identifying a pancreatic stem cell from the pancreas of a mammal, using a substance having specific affinity for each of 2 or more, preferably 3 or more, more preferably 4 or more, and most preferably all of the 5 marker proteins, selected from the group consisting of c-Met, c-Kit, CD45, TER119 and Flk-1, or a gene encoding the same.

In the present specification, the, “mammal” is concretely exemplified by human, bovine, horse, dog, guinea pig, mouse, rat and the like.

The c-Met protein is a protooncogene product, and functions as a hepatocyte growth factor receptor (HGF receptor) having tyrosine kinase activity. The relationship between the expression thereof and cancers in stomach, bile duct and the like has been reported. The c-Kit protein is also a similar protooncogene product, which is a receptor tyrosine kinase having a similar structure to that of a receptor of a macrophage colony-stimulating factor or a platelet-derived growth factor, and which has been reported to be involved in a certain kind of tumors. The CD45 protein is a kind of leukocyte common antigen, and is known to be expressed in all the hematopoietic cells except erythrocyte, platelet and progenitor cells thereof. The TER119 protein is known to be a cell surface marker effective for selecting erythroid cells. The Flk-1 protein is known to be an effective cell surface marker for selecting a vascular endothelial cell. The present invention is based on a new finding that these proteins and genes-encoding them show a unique expression pattern in a pancreatic stem cell. In the present specification, unless particularly specified, by the term “marker” is meant each member of the group consisting of a series of proteins showing such expression pattern unique to a pancreatic stem cell and genes encoding them. Such marker protein may have a different amino acid sequence depending on the kind of mammal and the like. In the present invention, as long as the expression pattern in the pancreatic stem cell is the same, such protein can be also used as a marker protein and is within the scope of the present invention. To be precise, a homologue having not less than 40% of homology in the amino acid sequence of each protein can be used as a marker protein in the present invention, namely, as c-Met, c-Kit, CD45, TER119 or Flk-1. The present invention comprises a step of analyzing the expression of such marker protein and/or a gene encoding the same, using a substance having specific affinity for each marker protein or a gene encoding the same.

In the present specification, the term “using” is not particularly limited as regards its method. Specifically, when a substance having specific affinity for, for example, a marker protein, is used, a method utilizing an antigen-antibody reaction with the antibody of said marker protein can be mentioned, and when a substance having specific affinity for a gene encoding a marker protein is used, a method utilizing a hybridization reaction can be mentioned (procedure is to be described in detail later).

As a substance having specific affinity for a marker protein, for example, an antibody having specific affinity for the protein, and a fragment thereof can be mentioned, where the specific affinity refers to an ability to specifically recognize a protein by an antigen-antibody reaction and to bind therewith. The antibody and a fragment thereof are not particularly limited as long as they can bind specifically with the protein, and may be a polyclonal antibody, a monoclonal antibody or a functional fragment thereof. These antibodies and their functional fragments can be produced according to a method generally employed in this field. For example, when a polyclonal antibody is used, a method wherein a protein is injected subcutaneously to the back of or intraperitoneally, intravenously and the like to an animal such as mouse and rabbit for immunization, and after awaiting an increase in the antibody titer, antiserum is harvested can be mentioned. When a monoclonal antibody is used, a method wherein a hybridoma is prepared according to a conventional method, and secretion therefrom is harvested can be mentioned. As a method of producing an antibody fragment, a method wherein a fragment of a cloned antibody gene is expressed in a microorganism and the like is frequently used. The purity of the antibody, antibody fragment and the like is not particularly limited as long as it retains specific affinity for the protein. These antibodies and fragments thereof may be labeled with a fluorescent substance, an enzyme a radioisotope and the like. Furthermore, commercially available ones may be used.

A substance having specific affinity for a gene encoding a marker protein is exemplified by oligo- or polynucleotide probe having specific affinity for the gene (hereinafter to be conveniently referred to simply as a probe), and an oligo- or polynucleotide primer pair having specific affinity for the gene (hereinafter to be conveniently referred to simply as a primer pair) can be mentioned. The “specific affinity” thereof refers to a property to hybridize only to the objective gene. Accordingly, it may be completely complementary to all or a part of the gene, or may include one to several mismatches as long as the above-mentioned property is satisfied. The probe and the primer pair are free of any particular limitation as long as they have specific affinity for the gene. For example, an oligo- or polynucleotide containing all or a part of the base sequence of the gene, a complementary sequence thereof, and the like can be mentioned, which are appropriately selected according to the form of the gene to be detected. The oligo- or polynucleotide is not particularly limited as to its origin as long as it has specific affinity for the gene. It may be a synthesized one or one obtained by cleaving out a necessary portion from the gene and purifying by a method generally employed. These oligo- and polynucleotides may be labeled with a fluorescence substance, an enzyme, a radioisotope and the like.

The separation or identification method of a pancreatic stem cell of the present invention is performed by analyzing at least 2, preferably 3, more preferably 4, most preferably all the 5 markers, using at least 2, preferably 3, more preferably 4, most preferably all 5 substances, from 5 kinds of substances having specific affinity for each of the above-mentioned five marker proteins, or a gene encoding the same. By such analysis, a pancreatic stem cell derived from a mammal can be obtained by separating a cell satisfying at least 2, preferably 3, more preferably 4, most preferably 5, of the characteristics of [c-Met expressing, c-Kit non-expressing, CD45 non-expressing, TER119 non-expressing, Flk-1 non-expressing] (also described as [c-Met⁺, c-Kit⁻, CD45⁻, TER119⁻, Flk-1⁻]). Such stem cell shows a [c-Met⁺, c-Kit⁻, CD45⁻, TER119⁻, Flk-1⁻] marker. As used herein, by the “shows” is meant that it has a characteristic of “expressing” or “non-expressing” each marker protein or a gene encoding the same.

As a method of selecting and separating a cell showing each marker using each substance having specific affinity, a method generally employed in this field or a combination of such methods is used. For example, for an analysis of a marker at a protein level, particularly when a live cell needs to be recovered, a method, wherein a pigment for labeling the substance is appropriately selected and flowcytometry is utilized, is convenient. More preferably, a cell is separated using a fluorescence-activated cell sorter (FACS). By the use of such device, the objective cell can be automatically separated and recovered.

When recovery of a live cell is not particularly necessary, such as in the case of identification and the like, the cell may be crushed, extracted, and mRNA is recovered and subjected to Northern blot. Alternatively, a membrane protein may be extracted and subjected to Western blot.

As a method of separating a stem cell using gene expression as an index, for example, a step comprising expression of a GFP (green fluorescent protein) gene using a c-Met promoter, and separation by FACS can be utilized. This step (separation and identification of c-Met expressing cell) combined with a method using expression of a different marker as an index becomes a more effective means of separating and identifying a stem cell.

In the present invention, using the separation method and identification method of the present invention, a pancreatic stem cell of a mammal can be obtained or confirmed. Such stem cell can be differentiated into a cell having various physiological functions by induction of differentiation under suitable conditions (hereinafter a cell that has come to exert a physiological function by differentiation and induction in this manner is to be conveniently referred to as a functional cell). As used herein, by the “under suitable conditions” is meant being under culture conditions with the addition of cytokines such as a hepatocyte growth factor (HGF), an epidermal growth factor (EGF) and the like. For example, a pancreatic stem cell is known to differentiate into an α cell, which is a glucagon-producing cell, a β cell which is an insulin-producing cell, a γ cell, which is a pancreatic polypeptide-producing cell, a δ cell, which is a somatostatin-producing cell, an exocrine cell and the like, and such differentiation can be confirmed by examining the presence or absence of a physiologically active substance these cells express and secrete.

According to the present invention, since a stem cell can be supplied stably, a cell differentiated from the stem cell can be also provided at a desired time in a desired amount. For example, it is expected that, by implanting a cell differentiated from a pancreatic stem cell into the body of a patient suffering from a pancreatic hypofunctional disease, the pancreatic function can be recovered and a therapeutic effect on a pancreatic hypofunctional disease can be afforded. As a pancreatic hypofunctional disease, diabetes, chronic pancreatitis, autoimmune pancreatitis, pancreatic functional disorder observed after removal of pancreas and the like can be mentioned. In addition, since a cell differentiated from a pancreatic stem cell of the present invention expresses a liver•bile duct or stomach•intestine marker, it is expected that, by implanting a cell differentiated from a pancreatic stem cell into the body of a patient suffering from a liver•bile duct or stomach•intestinal hypofunctional disease, the liver•bile duct or stomach•intestinal function can be recovered, and a therapeutic effect on a liver•bile duct or stomach•intestinal hypofunctional disease can be afforded. As a liver•bile duct hypofunctional disease, acute hepatitis, chronic hepatitis, metabolic liver disease and the like can be mentioned, and as a stomach•intestinal hypofunctional disease, short bowel syndrome, inflammatory bowel disease, functional disorder of stomach observed after removal of the stomach and the like can be mentioned.

The present invention further provides a method of screening a substance that induces differentiation of a pancreatic stem cell, using the pancreatic stem cell of the present invention. This method comprises at least the following steps:
(1) a step of reacting a pancreatic stem cell with a test substance, and
(2) a step of determining an expression of a pancreatic marker in the-cell after the reaction.

The pancreatic stem cell to be used in the present screening method is not particularly limited as long as it differentiates into a functional cell of pancreas, which is preferably a pancreatic stem cell separated by the aforementioned method of the present invention. While the reaction between the stem cell and a test substance is appropriately set depending on the desired kind of differentiation, differentiation inducing conditions and the like, culture is generally performed at 35-40° C. for several min to several dozen days, preferably at 36.5-37.5° C. for 8 to 30 days. After the reaction with the test substance, the presence or absence and the state of the differentiation of the stem cell are examined.

Whether or not the stem cell has differentiated can be confirmed by determining the expression of the pancreatic marker. The marker is appropriately selected according to a desired functional cell. As used herein, the pancreatic marker is a protein that is expressed and produced (secreted) in a pancreas specific manner, particularly specifically to the functional cell of the pancreas, or a gene encoding the same. For example, the induction ability of differentiation into α cell can be confirmed by examining the production and secretion of glucagon and the induction ability of differentiation into β cell can be confirmed by examining the production and secretion of insulin.

The present invention moreover provides a method of screening a substance that regulates the function of pancreas using the pancreatic stem cell of the present invention or a functional cell differentiated from the stem cell by induction. This method comprises at least the following steps:.
(1) a step of reacting a pancreatic stem cell or a cell differentiated from the stem cell with a test substance, and
(2) a step of determining the expression of a pancreatic marker in the cell after the reaction.

The pancreatic stem cell and a functional cell differentiated from the stem cell to be used for this screening method are not particularly limited as long as they are a cell to be differentiated into a functional cell of the pancreas and a cell differentiated from the cell by induction, and are preferably a pancreatic stem cell separated by the aforementioned method of the present invention and a cell differentiated therefrom under suitable conditions (mentioned above). For example, the screening is performed concretely as follows. A test substance is added to a culture system of a pancreatic stem cell, and after culture for 8-30 days, expression of a gene or a protein (marker) in a functional cell differentiated by induction is analyzed, whereby the role of the test substance in the differentiation and proliferation of the pancreatic functional cell is analyzed (quantitative PCR, Northern blotting, immunostaining, ELISA etc.).

As a test substance to be the target in the screening method of the present invention, various known and unknown substances are mentioned. Specifically, a known cytokine, an extracellular matrix, an inorganic compound, a culture supernatant of a suitable cultured cell line, an unknown gene obtained from a suitable cDNA library, a recombinant protein of the gene and the like can be mentioned.

The present invention further provides a method of screening a substance that induces differentiation into the liver•bile duct or stomach•intestine using a pancreatic stem cell of the present invention. This method comprises at least the following steps:
(1) a step of reacting the pancreatic stem cell of the present invention with a test substance, and
(2) a step of determining the expression of a liver•bile duct or stomach•intestine marker in the cell after the reaction.

While the reaction between the stem cell and a test substance is appropriately set depending on the desired kind of differentiation, differentiation-inducing conditions and the like, culture is generally performed at 35-40° C. for several min to several dozen days, preferably at 36.5-37.5° C. for 8 to 30 days. After the reaction with the test substance, the presence or absence and the state of the differentiation of the stem cell are examined.

Whether or not the stem cell has differentiated can be confirmed by determining the expression of the liver•bile duct or stomach•intestine marker. The marker is appropriately selected according to a desired functional cell. As used herein, the liver•bile duct or stomach•intestine marker is a protein that is expressed or produced (secreted) in a liver•bile duct or stomach•intestine specific manner, particularly specifically to the functional cell of the liver•bile duct or stomach•intestine, or a gene encoding the same. For example, the liver•bile duct cell marker includes albumin, α-fetoprotein, glucose-6-phosphatase, transthyretin, cytokeratin 19, cytokeratin 18, cytokeratin 8, biliary glycoprotein, and a gene encoding the same, and the stomach•intestine cell marker includes fabp-2, GIP, CCK, TPH, pepsinogen F, gastrin and a gene encoding the same.

The present invention moreover provides a method of screening a substance that regulates the function of the liver•bile duct or stomach•intestinal using the pancreatic stem cell of the present invention or a functional cell differentiated from the stem cell by induction. This method comprises at least the following steps:
(1) a step of reacting the pancreatic stem cell of the present invention or a cell differentiated from the stem cell with a test substance, and
(2) a step of determining the expression of a liver•bile duct or stomach•intestine marker in the cell after the reaction.

For example, the screening is concretely performed as follows. A test substance is added to the-culture system of a pancreatic stem cell, and after culture for 8-30 days, the expression of a gene or a protein (marker) in a functional cell differentiated from the stem cell by induction is analyzed, whereby the role of the test substance in the differentiation and proliferation of the pancreatic functional cell is analyzed (quantitative PCR, Northern blotting, immunostaining, ELISA etc.).

As a test substance to be the target in the screening method of the present invention, various known and unknown substances are mentioned. Specifically, a known cytokine, an extracellular matrix, an inorganic compound, a culture supernatant of a suitable cultured cell line, an unknown gene obtained from a suitable cDNA library, a recombinant protein of the gene and the like can be mentioned.

The pancreatic stem cell differentiation-inducing substance, a substance that induces differentiation into the liver•bile duct or stomach•intestine, a pancreatic function regulating substance, and a liver•bile duct or stomach•intestinal function regulating substance, which are obtained by such a screening method, are useful as therapeutic drugs of a pancreatic hypofunctional disease, liver•bile duct hypofunctional disease, and stomach•intestinal hypofunctional disease. Here, the pancreatic hypofunctional disease, liver•bile duct hypofunctional disease, and stomach•intestinal hypofunctional disease are as mentioned above.

A pharmaceutical agent or composition containing the pancreatic stem cell of the present invention or a cell differentiated from the stem cell (hereinafter to be referred to as the pharmaceutical agent or composition of the present invention) is effective for the prophylaxis or treatment of pancreatic hypofunctional diseases, liver•bile duct hypofunctional diseases and stomach•intestinal hypofunctional diseases in various mammals such as human, monkey, mouse, rat, rabbit, pig, dog, horse, bovine and the like. While the dose is not particularly limited as long as it is an amount necessary for the treatment of the disease, it varies depending on the conditions (species, age, sex, body weight and the like) and symptoms of the subject in need of such treatment.

The pharmaceutical agent or composition of the present invention can contain a conventional pharmaceutically acceptable and substantially non-toxic carrier or excipient used for, for example, tablet, pellet, troche, capsule, suppository, cream, ointment, aerosol, powder preparation for inhalation, liquid, emulsion, suspension, other dosage forms suitable for use. In addition, an additive such as aid, stabilizer, thickener, colorant, flavor and the like can be added where necessary.

The pharmaceutical agent or composition of the present invention can be produced using a preparation technique known in the pertinent field. The pharmaceutical agent or composition of the present invention can be converted to a prodrug as necessary by a method known in this field.

### EXAMPLES

While the present invention is concretely explained in detail in the following by referring to Examples, the present invention is not limited in any way by these examples.

### Example 1

Recovery of Pancreatic Cells

The pancreas was removed from C57BL/6 mouse neonate (one day after birth) under a stereo microscope, and placed in a Ca²⁺ free Hank's balanced salt solution containing 0.05% collagenase and 5 mmol/L CaCl₂ (pH 7.4). This was incubated at 37° C. for 10 min and gently pipetting to isolate the cells, which cells were washed 3 times with a cell culture medium. After preparation, not less than 80% of cells were viable by detection with Trypan blue. As the cell culture medium, one having the following composition was independently prepared and used.

(Preparation of Cell Culture Medium)

To a 1:1 mixture of DMEM and F-12 were prepared by adding fetal bovine serum (FBS, 10%), γ-insulin (1 μg/mL), dexamethasone (1×10⁻⁷ M), nicotinamide (10 mM), L-glutamine (2 mM), β-mercaptoethanol (50 μM), HEPES (5 mM) and penicillin/streptomycin.

Flowcytometry

The prepared cells were first reacted with a biotinylated anti-CD45 monoclonal antibody (PharMingen, San Jose, Calif.), a biotinylated anti-TER119 monoclonal antibody (PharMingen, San Jose, Calif.), and an anti-c-Met monoclonal antibody (Upstate biotechnology, Lake Placid, N.Y.) in ice water for 30 min. After the reaction, the cells were washed 3 times with a staining medium (PBS (phosphate-buffered saline) containing 3% FBS) and subsequently reacted with a FITC (fluorescein isothiocyanate)-labeled anti-mouse IgG2a monoclonal antibody (PharMingen, San Jose, Calif.), an APC (allophycocyanin)-labeled anti-c-Kit monoclonal antibody (PharMingen, San Jose, Calif.), and a Texasred-labeled streptavidin (Gibco BRL, GaithersBurg, Md.) in ice water for 30 min. Finally, the cells were washed 3 times with a staining medium and suspended in a staining medium containing PI (propidium iodide) (5 μg/mL). These fluorescence-labeled cells were analyzed by FACS-vantage and separated into various fractions. The gate was set with the negative control as an index.

Recovery of Cells

CD45⁻, TER119⁻ cells were gated to remove. CD45⁺ cell and TER119⁺ cell. By this operation, blood cells are removed. Then, CD45⁻, TER119⁻ cells-were fractionated into c-Met⁺/c-Kit⁻, c-Met⁻/c-Kit⁺, c-Met⁺/c-Kit⁺ and c-Met⁻/c-Kit⁻ cell fractions and a cell recovery gate was set. Subsequently, the recovered cells were sown on a 35 mm diameter tissue culture dish at a concentration of 200 cells/cm² for low density culture. The residual erythrocytes, wreck of the cells, cell aggregation of 2 or more agglutinated cells and dead cells were removed by forward scattering, side scattering and PI. After the recovery, not less than 90% of the cells were viable by the detection with Trypan blue.

In Vitro Colony Assay

The cells recovered by FACS were cultured in the above-mentioned cell culture medium (37° C., 5% CO₂). After 24 hrs from the start of the culture, human recombinant hepatocyte growth factor (rHGF) (50 ng/mL; Sigma) and epidermal growth factor (EGF) (20 ng/mL; Sigma) were added. The number of colonies was counted at day 8 from the start of the culture.

RT-PCR

The colonies were culture for 14 days and applied a cloning ring thereon, into which ISOGEN was added and gently pipetted, whereby RNA was obtained from a colony-forming cell. Subsequently, cDNA was prepared from the obtained RNA using reverse transcriptase (Super Script II). Then, using the prepared cDNA as a template and the following primer, PCR was conducted to try detection of the markers of various pancreatic functional cells.

The objective markers and their primers are shown in Table 1.

Each primer shown in Table 1 was ordered from Hokkaido System Science CO., LTD and synthesized for use.

The PCR cycle was 95° C. (4 min)→[94° C. (1 min)→56° C. (1 min)→72° C. (1 min)×45 cycles→72° C. (10 min). The nucleic acid produced by. PCR was developed with 2% agarose gel and analyzed.

Immunostaining

The colonies formed at day 14 of culture were washed 3 times with PBS, fixed with methanol (−20° C., 10 min) and washed 3 times with PBS containing Tween 20 (0.05%) (PBS-Tween 20). After blocking with 10% monkey serum, the colonies were reacted (4° C., 16 hrs) with a goat anti-insulin antibody or a mouse anti-glucagon antibody. After washing 3 times with PBS-Tween 20, blocking was performed again, and then the colonies were reacted (40C, 3 hrs) respectively with an Alexa 488-labeled monkey anti-goat IgG antibody and a Cy3-labeled monkey anti-mouse IgG antibody. Finally, the colonies were washed 3 times with PBS-Tween 20, observed under Zeiss LSM510 laser scanning microscope and analyzed.

Results

Pancreatic cells prepared from a mouse neonate were gated using FACS for viable cells (ca. 80%) and developed sing antibodies against CD45 and TER119, respectively, to clarify the [CD45⁻, TER119⁻] cell fraction (ca. 40% of the whole), which was a non-blood cell fraction. Then, [CD45⁻, TER119⁻] cells were further developed with antibodies against c-Met (HGF receptor) and c-Kit (SCF receptor), respectively. The [c-Met⁺, c-Kit⁺, CD45⁻, TER119⁻] cells were about 0.01% of the whole, the. [c-Met⁺, c-Kit⁻, CD45⁻, TER119⁻] cells were about 1.0% of the whole, the [c-Met⁻, c-Kit⁺, CD45⁻, TER119⁻] cells were about 0.8% of the whole, and the [c-Met⁻, c-Kit⁻, CD45⁻, TER119⁻] cells were about 36% of the whole. The cells contained in each fraction were recovered in a viable state and cultured (200 cells/cm²), and colonies were assayed in vitro. Some of the recovered cells showed high proliferation ability and they form a single cell-derived clonal colony. By observation at day 8 of culture, two kinds of colonies formed by morphologically distinct cells were mainly found. The colony formed by the epithelial cells was named an epithelial colony and the colony like a fibroblast was named a fibroblast-like colony. cDNA was separately prepared from the distinct colonies and the markers of various pancreatic functional cells were detected by PCR. As a result, pancreatic functional cell markers, such as insulin and glucagon, were detected only in epithelial colonies. Accordingly, it was strongly suggested that pancreatic stem/progenitor cells were contained in the epithelial colony-forming cells (epithelial colony-forming cell: ECFC). The ECFC was counted and, as a result, it was clarified that ECFC was confined to a [c-Met⁺, c-Kit⁻, CD45⁻, TER119⁻] cell fraction and present at high frequency (13.1-fold as compared to non-gated cells, and 9.45-fold as compared to [CD45⁻, TER119⁻] cell fraction).

Then, whether or not these ECFCs have multi-differentiation ability, which is among the characteristics of the pancreatic stem/progenitor cells, was analyzed by RT-PCR method and immunostain method. A total of 30 clonal colonies were analyzed by RT-PCR method, and as a result, many colonies in these colonies contained all or some of an α cell which is a glucagon-producing cell, β cell which is an insulin-producing cell, a γ cell which is a pancreatic polypeptide-producing cell and a δ cell which is a somatostatin-producing cell, and a number of colonies containing a cell that expresses amylase or lipase, each of which is an exocrine cell marker, were also observed. Similarly, when the immunostain method was used, the co-presence of an α cell which is a glucagon-producing cell, and a β cell which is an insulin-producing cell, was observed in the same colony. From these results, it was clarified that a cell (ECFC)-having a multi-differentiation ability, which is one of the important properties of a pancreatic stem/progenitor cell, is concentrated highly frequently in a [c-Met⁺, c-kit⁻, CD45⁻, TER119⁻ cell fraction occupying only 1% of the mouse neonate pancreatic cells. It was simultaneously clarified that these cells can be separated and recovered alive using FACS.

### Example 2

Recovery of Pancreatic Cells

Pancreatic cells were recovered-in the same manner as in Example 1. The cell culture medium used was the same as the one used in Example 1.

Flowcytometry

Conducted according to a similar method as in Example 1 except that in addition to the various labeled antibodies and labeling reagents used in Example 1, PE (phycoerythrin)-labeled anti-Flk-1 monoclonal antibody (PharMingen, San Jose, Calif.) was also used.

Recovery of Cells

Conducted according to a similar method as in Example 1 except that the [c-Met⁺, c-Kit⁻, CD45⁻, TER119⁻] cells obtained in the same manner as in Example 1 were further fractionated into Flk-1 positive cells and negative cells.

In Vitro Colony Assay

Conducted according to a similar method as in Example 1.

RT-PCR

Conducted according to a similar method as in Example 1.

Immunostaining

Conducted according to a similar method as in Example 1.

Results

The [c-Met⁺, c-Kit⁻, CD45⁻, TER119⁻] cells obtained in Example 1 were fractionated into Flk-1 positive and negative cells. The [c-Met⁺, c-Kit⁻, CD45⁻, TER119⁻, Flk-1] cells were about 0.7% of the whole cells (all recovered pancreatic cells) and the [c-Met⁺, c-Kit⁻, CD45⁻, TER119⁻, Flk-1⁺] cells were about 0.3% of the whole cells. In the same manner as in Example 1, ECFC was counted. As a result, it was clarified that ECFC was further limited and contained at higher frequency in the [c-Met⁺, c-Kit⁻, CD45⁻, TER119⁻, Flk-1⁻] cell fraction, as compared to the (c-Met⁺, c-Kit⁻, CD45⁻, TER119⁻] cell fraction (19.7-fold as compared to non-gated cells, 14.1-fold as compared to [CD45⁻, TER119⁻] cell fraction).

Then, whether or not these ECFCs have multi-differentiation ability, which is among the characteristics of the pancreatic stem/progenitor cells, was analyzed by RT-PCR method and immunostaining method in the same manner as in Example 1. As a result, it was clarified that a cell (ECFC) having a multi-differentiation ability, which is one of the important properties of a pancreatic stem/progenitor cell, is concentrated at high frequently in a [c-Met⁺, c-Kit⁻, CD45⁻, TER119⁻, Flk-1⁻] cell fraction occupying only. 0.7% of the mouse neonate pancreatic cells. It was simultaneously clarified that these cells could be separated and recovered alive using FACS.

### Example 3

Recovery of Pancreatic Cells

Pancreatic cells were recovered in the same manner as in Example 1. The cell culture medium was the same as the one used in Example 1.

Flowcytometry

Conducted according to a similar method as in Example 1 except that, in addition to the various labeled antibodies and labeling reagents used in Example 1, PE (phycoerythrin)-labeled anti-Flk-1 monoclonal antibody (PharMingen, San Jose, Calif.) was also used.

Recovery of Cells

Conducted according to a similar method as in Example 1 except the [c-Met⁺, c-Kit⁻, CD45⁻, TER119⁻] cells obtained in the same manner as in Example 1 were further fractionated into Flk-1 positive cells and negative cells.

In Vitro Culture

The cells recovered by FACS were cultured in the same cell culture medium as in Example 1 (37° C., 5% CO₂). At 24 hrs from the start of the culture, human recombinant hepatocyte growth factor (rHGF)(50 ng/mL; Sigma);and epidermal growth factor (EGF) (20 ng/mL; Sigma) were added. The cells were isolated by flowcytometry at day 30 from the start of the culture, and expression of the differentiation marker in the cells contained in the colony formed thereby was analyzed by RT-PCR.

RT-PCR

Conducted according to a similar method as in Example 1.

Results

The [c-Met⁺; c-Kit⁻, CD45⁻, TER119⁻, Flk-1⁻] cells were cultured for 30 days; isolated by flowcytometry and the expression of differentiation markers in the cells contained in the colony formed thereby was analyzed. As a result, the expression of albumin, α-fetoprotein, glucose-6-phosphatas, transthyretin, cytokeratin 19, cytokeratin 18, cytokeratin 8 and biliary glycoprotein as liver•bile duct cell markers, and the expression of fabp-2, GIP, CCK, TPH, pepsinogen F and gastrin as stomach•intestine cell markers was observed, in addition to various pancreatic cell markers.

TABLE 1

| marker | | primer |
|---|---|---|
| α cell | preproglucagon | 5′-ATTTACTTTGTGGCTGGATTG-3′ |
| specific | | (sequence No. 1) |
| marker | | 5′-TGTCAGTGATCTTGGTTTGAA-3′ |
| | | (sequence No. 2) |
| β cell | preproinsulin I | 5′-CTGTTGGTGCACTTCCTACC-3′ |
| specific | | (sequence No. 3) |
| marker | | 5′-GCAGTAGTTCTCCAGCTGGT-3′ |
| | | (sequence No. 4) |
| | preproinsulin II | 5′-TCAAGCAGCACCTTTGTGGTT-3′ |
| | | (sequence No. 5) |
| | | 5′-GTTGCAGTAGTTCTCCAGCTG-3′ |
| | | (sequence No. 6) |
| γ cell | pancreatic | 5′-CTCCCTGTTTCTCGTATCCA-3′ |
| specific | polypeptide | (sequence No. 7) |
| marker | | 5′-AGAGCAGGGAATCAAGCCAA-3′ |
| | | (sequence No. 8) |
| δ cell | preprosomatostatin | 5′-CTCTGCATCGTCCTGGCTT-3′ |
| specific | | (sequence No. 9) |
| marker | | 5′-CAGGATGTGAATGTCTTCCAG-3′ |
| | | (sequence No. 10) |
| exocrine | amylase 2 | 5′-AGTACCTGTGGAAGTTACCT-3′ |
| cell | | (sequence No. 11) |
| specific | | 5′-ACACAAGGGCTCTGTCAGAA-3′ |
| marker | | (sequence No. 12) |
| | hormone-sensitive | 5′-TCTTCTTCGAGGGTGATGAA-3′ |
| | lipase | (sequence No. 13) |
| | | 5′-TACCTTGCTGTCCTGTCCTT-3′ |
| | | (sequence No. 14) |
| Others | ipf1/pdx1 | 5′-TTACAAGCTCGCTGGGATCAC-3′ |
| | | (sequence No. 15) |
| | | 5′-AGGTCACCGCACAATCTTGCT-3′ |
| | | (sequence No. 16) |
| | c-met | 5′-AGCCAGTAATGATCTCAATG-3′ |
| | | (sequence No. 17) |
| | | 5′-TCAGGATAGGGGACAGGT-3′ |
| | | (sequence No. 18) |
| Positive | hypoxanthine | 5′-CTGTAATGATCAGTCAACGGC-3′ |
| control | phosphoribosyl | (sequence No. 19) |
| | transferase (HPRT) | 5′-GGCCTATAGGCTCATAGTGCA-3′ |
| | | (sequence No. 20) |
| liver- | albumin | 5′-CATGACACCATGCCTGCTGAT-3′ |
| bile duct | | (sequence No. 21) |
| cell | | 5′-CTCTGATCTTCAGGAAGTGTAC-3′ |
| marker | | (sequence No. 22) |
| | α-fetoprotein | 5′-ACTCACCCCAACCTTCCTGTC-3′ |
| | | (sequence No. 23) |
| | | 5′-CAGCAGTGGCTGATACCAGAG-3′ |
| | | (sequence No: 24) |
| | glucose-6- | 5′-AACCCATTGTGAGGCCAGAGG-3′ |
| | phosphatase | (sequence No. 25) |
| | | 5′-TACTCATTACACTAGTTGGTC-3′ |
| | | (sequence No. 26) |
| | transthyretin | 5′-TGGTATTTGTGTCTGAAGCTG-3′ |
| | | (sequence No. 27) |
| | | 5′-TTAATAAGAATGCTTCACGGC-3′ |
| | | (sequence No. 28) |
| | cytokeratin 19 | 5′-GTCCTACAGATTGACAATGC-3′ |
| | | (sequence No. 29) |
| | | 5′-CACGCTCTGGATCTGTGACAG-3′ |
| | | (sequence No.30) |
| | cytokeratin 18 | 5′-GGACCTCAGCAAGATCATGGC-3′ |
| | | (sequence No.31) |
| | | 5′-CCACGATCTTACGGGTAGTTG-3′ |
| | | (sequence No.32) |
| | cytokeratin 8 | 5′-AGTCTCAGATCTCAGACACG-3′ |
| | | (sequence No. 33) |
| | | 5′-CCATAGGATGAACTCAGTCC-3′ |
| | | (sequence No. 34) |
| | biliary glycoprotein | 5′-GAACTAGACTCTGTCACCCTG-3′ |
| | | (sequence No. 35) |
| | | 5′-GCCAGACTTCCTGGAATAGA-3′ |
| | | (sequence No. 36) |
| stomach - | intestinal fatty | 5′-ATTCGACGGCACGTGGAAAGT-3′ |
| intestine | acid binding | (sequence No. 37) |
| cell | protein; fabp-2 | 5′-AAGAATCGCTTGGCCTCAACT-3′ |
| marker | | (sequence No. 38) |
| | gastric inhibitory | Jensen J., Pedersen E. E., |
| | peptide (GIP) | Galante P., Hald J., Heller |
| | | R. S., Ishibashi M., Kageyama |
| | | R., Guillemot F., Serup P., |
| | | and Madsen O. D. 2000. Control |
| | | of endodermal endocrine |
| | | development by Hes-1. Nat. |
| | | Genet. 24: 36-44. |
| | cholecystokinin (CCK) | Jensen et al., supra. |
| | tryptophan | Jensen et al., supra. |
| | hydroxylase (TPH) | |
| | pepsinogen F | 5′-ACCTAGACCTGGTCTACATTG-3′ |
| | | (sequence No. 39) |
| | | 5′-AGTGAAGCTCTCCATGGTAGT-3′ |
| | | (sequence No. 40) |
| | gastrin | Jensen et al., supra. |

Industrial Applicability

The pancreatic stem cell of the present invention and various functional cells differentiated from the stem cell recover pancreas function by implanting the cells into the body of patients with pancreatic hypofunctional disease, such as diabetes and the like, and are expected to show-a treatment effect on diabetes and the like. Moreover, a treatment effect on liver•bile duct or stomach•intestinal hypofunctional diseases can be also expected.

Furthermore, by the use of the stem cell and/or the functional cell, a substance that induces differentiation of pancreatic stem cell, a substance that induces differentiation into liver•bile duct or stomach•intestine, a pancreatic function regulating substance, and a liver•bile duct or stomach•intestinal function regulating substance can-be screened. Such substance is promising as a therapeutic agent of pancreatic, liver•bile duct or stomach•intestinal hypofunctional diseases.

This application is based on a patent application No. 2001-126315 filed in Japan, the contents of which are hereby incorporated by reference.

Sequence Listing Free Text

Sequence No. 1: oligonucleotide designed to act as a PCR primer for detecting preproglucagon (α cell specific marker) gene.
Sequence No. 2: oligonucleotide designed to act as a PCR primer for detecting preproglucagon (α cell specific marker) gene.
Sequence No. 3: oligonucleotide designed to act as a PCR primer for detecting preproinsulin I (β cell specific marker) gene.
Sequence No. 4: oligonucleotide designed to act as a PCR primer for detecting preproinsulin I (β cell specific marker) gene.
Sequence No. 5: oligonucleotide designed to act as a PCR primer for detecting preproinsulin II (β cell specific marker) gene.
Sequence No. 6: oligonucleotide designed to act as a PCR primer for detecting preproinsulin II (β cell specific marker) gene.
Sequence No. 7: oligonucleotide designed to act as a PCR primer for detecting pancreatic polypeptide (γ cell specific marker) gene.
Sequence No. 8: oligonucleotide designed to act as a PCR primer for detecting pancreatic polypeptide (γ cell specific marker) gene.
Sequence No. 9: oligonucleotide designed to act as a PCR primer for detecting preprosomatostatin (δ cell specific marker) gene.
Sequence No. 10: oligonucleotide designed to act as a PCR primer for detecting preprosomatostatin (δ cell specific marker) gene.
Sequence No. 11: oligonucleotide designed to act as a PCR primer for detecting amylase 2 (exocrine cell-specific marker) gene.
Sequence No. 12: oligonucleotide designed to act as a PCR primer for detecting amylase 2 (exocrine cell specific marker) gene.
Sequence No. 13: oligonucleotide designed to act as a PCR primer for detecting hormone-sensitive lipase (exocrine cell specific marker) gene.
Sequence No. 14: oligonucleotide designed to act as a PCR primer for detecting hormone-sensitive lipase (exocrine cell specific marker) gene.
Sequence No. 15: oligonucleotide designed to act as a PCR primer for detecting ipf1/pdx1 gene.
Sequence No. 16: oligonucleotide designed to act as a PCR primer for detecting ipf1/pdx1 gene.
Sequence No. 17: oligonucleotide designed to act as a PCR primer for detecting c-Met gene.
Sequence No. 18: oligonucleotide designed to act as a PCR primer for detecting c-Met gene.
Sequence No. 19: oligonucleotide designed to act as a PCR primer for detecting Hypoxanthine Phosphoribosyl Transferase (HPRT, positive control) gene.
Sequence No. 20: oligonucleotide designed to act as a PCR primer for detecting Hypoxanthine Phosphoribosyl Transferase (HPRT, positive control) gene.
Sequence No. 21: oligonucleotide designed to act as a PCR primer for detecting albumin (liver•bile duct cell marker) gene.
Sequence No. 22: oligonucleotide designed to act as a PCR primer for detecting albumin (liver•bile duct cell marker) gene.
Sequence No. 23: oligonucleotide designed to act as a PCR primer for detecting α-fetoprotein (liver•bile duct cell marker) gene.
Sequence No. 24: oligonucleotide designed to act as a PCR primer for detecting α-fetoprotein (liver•bile duct cell marker) gene.
Sequence No. 25: oligonucleotide designed to act as a PCR primer for detecting glucose-6-phosphatase (liver•bile duct cell marker) gene.
Sequence No. 26: oligonucleotide designed to act as a PCR primer for detecting glucose-6-phosphatase (liver•bile duct cell marker) gene.
Sequence No. 27: oligonucleotide designed to act as a PCR primer for detecting Transthyretin (liver•bile duct cell marker) gene.
Sequence No. 28: oligonucleotide designed to act as a PCR primer for detecting transthyretin (liver•bile duct cell marker) gene.
Sequence No. 29: oligonucleotide designed to act as a PCR primer for detecting cytokeratin 19 (liver•bile duct cell marker) gene.
Sequence No. 30: oligonucleotide designed to act as a PCR primer for detecting cytokeratin 19 (liver•bile duct cell marker) gene.
Sequence No. 31: oligonucleotide designed to act as a PCR primer for detecting cytokeratin 18 (liver•bile duct cell marker) gene.
Sequence No. 32: oligonucleotide designed to act as a PCR primer for detecting cytokeratin 18 (liver•bile duct cell marker) gene.
Sequence No. 33: oligonucleotide designed to act as a PCR primer for detecting cytokeratin 8 (liver•bile duct cell marker) gene.
Sequence No. 34: oligonucleotide designed to act as a PCR primer for detecting cytokeratin 8 (liver•bile duct cell marker) gene.
Sequence No. 35: oligonucleotide designed to act as a PCR primer for detecting biliary glycoprotein (liver•bile duct cell marker) gene.
Sequence No. 36: oligonucleotide designed to act as a PCR primer for detecting biliary glycoprotein (liver•bile duct cell marker) gene.
Sequence No. 37: oligonucleotide designed to act as a PCR primer for detecting intestinal fatty acid binding protein (stomach•intestine cell marker) gene.
Sequence No. 38: oligonucleotide designed to act as a PCR primer for detecting intestinal fatty acid binding protein (stomach•intestine cell marker) gene.
Sequence No. 39: oligonucleotide designed to act as a PCR primer for detecting pepsinogen F (stomach•intestine cell marker) gene.
Sequence No. 40: oligonucleotide designed to act as a PCR primer for detecting pepsinogen F (stomach•intestine cell marker) gene.

1. A method of separating a pancreatic stem cell from the pancreas of a mammal using two or more kinds of substances having specific affinity for a marker protein selected from the group consisting of c-Met, c-Kit, CD45 and TER119, or a gene encoding the same.

2. A method of separating a pancreatic stem cell from the pancreas of a mammal using two or more kinds of substances having specific affinity for a marker protein selected from the group consisting of c-Met, c-Kit, CD45, TER119 and Flk-1, or a gene encoding the same.

3. The method of claim 1, wherein the substance having specific affinity is an antibody against the marker protein.

4. A method of identifying a pancreatic stem cell of a mammal using two or more kinds of substances having specific affinity for a marker protein selected from the group consisting of c-Met, c-Kit, CD45 and TER119, or a gene encoding the same.

5. A method of identifying a pancreatic stem cell of a mammal using two or more kinds of substances having specific affinity for a marker protein selected from the group consisting of c-Met, c-Kit, CD45, TER119 and Flk-1, or a gene encoding the same.

6. The method of claim 4, wherein the substance having specific affinity is an antibody against the marker protein.

7. A method of separating a pancreatic stem cell from the pancreas of a mammal, which comprises a step of analyzing the expression state of two or more marker proteins selected from the group consisting of c-Met, c-Kit, CD45 and TER119, or a gene encoding the same.

8. A method of separating a pancreatic stem cell from the pancreas of a mammal, which comprises a step of analyzing the expression state of two or more marker proteins selected from the group consisting of c-Met, c-Kit, CD45, TER119 and Flk-1, or a gene encoding the same.

9. A method of identifying a pancreatic stem cell from the pancreas of a mammal, which comprises a step of analyzing the expression state of two or more marker proteins selected from the group consisting of c-Met, c-Kit, CD45 and TER119, or a gene encoding the same.

10. A method of identifying a pancreatic stem cell from the pancreas of a mammal, which comprises a step of analyzing the expression state of two or more marker proteins selected from the group consisting of c-Met, c-Kit, CD45, TER119 and Flk-1, or a gene encoding the same.

11. A pancreatic stem cell that can be separated from the pancreas of a mammal by the method described in claim 1.

12. The pancreatic stem cell of claim 11, which shows 4 markers of c-Met, c-Kit, CD45 and TER119 in a pattern of c-Met⁺, c-Kit⁻, CD45⁻, TER119⁻.

13. The pancreatic stem cell of claim 11, which shows 5 markers of c-Met, c-Kit, CD45, TER119 and Flk-1 in a pattern of c-Met⁺, c-Kit⁻, CD45⁻, TER119⁻, Flk-1⁻.

14. A method of screening a substance that induces differentiation of a pancreatic stem cell of a mammal, which comprises the following steps:
(i) a step of reacting a pancreatic stem cell with a test substance, and
(ii) a step of determining the expression of a pancreatic marker in the cell after the reaction.

15. A method of screening a substance that induces differentiation into liver•bile duct or stomach•intestine of a mammal, which comprises the following steps:
(i) a step of reacting a pancreatic stem cell of claim 12 with a test substance, and
(ii) a step of determining the expression of a liver•bile duct or stomach•intestine marker in the cell after the reaction.

16. A method of screening a substance that regulates a pancreatic function of a mammal, which comprises the following steps:
(i) a step of reacting a pancreatic stem cell or a cell differentiated from the stem cell with a test substance, and
(ii) a step of determining the expression of a pancreatic marker in the cell after the reaction.

17. A method of screening a substance that regulates the function of liver•bile duct or stomach•intestine of a mammal, which comprises the following steps:
(i) a step of reacting a pancreatic stem cell of claim 12 or a cell differentiated from the stem cell with a test substance, and
(ii) a step of determining the expression of a liver•bile duct or stomach•intestine marker in the cell after the reaction.

18. A cloned pluripotent pancreatic stem cell, which satisfies at least 2 of the characteristics selected from the group consisting of c-Met⁺, c-Kit⁻, CD45⁻ and TER119⁻.

19. A cloned pluripotent pancreatic stem cell, which satisfies at least 2 of the characteristics selected from the group consisting of c-Met⁺, c-Kit⁻, CD45⁻, TER119⁻ and Flk-1⁻.

20. A pharmaceutical composition, which comprises:
i) a cloned pluripotent pancreatic stem cell of claim 18 and
ii) a pharmaceutically acceptable carrier.

21. A purified composition, which comprises: a cloned pluripotent pancreatic stem cell of claim 18.

22. Tissue regenerated from a cloned pluripotent pancreatic stem cell of claim 18.

23. An organ regenerated from a cloned pluripotent pancreatic stem cell of claim 18.

24. A method of transplanting a cloned pluripotent pancreatic stem cell into a host, which comprises:
i) obtaining the cloned pluripotent pancreatic stem cell of claim 18; and
ii) transplanting said stem cell into the host.

25. A method of producing a pancreatic stem cell, which comprises:
i) providing cells from the pancreas of a mammal; and
ii) selecting cells which satisfies at least 2 of the marker protein expression patterns selected from the group consisting of c-Met⁺, c-Kit⁻, CD45⁻ and TER119⁻, or a gene encoding the same.

26. A method of producing a pancreatic stem cell, which comprises:
i) providing cells from the pancreas of a mammal; and
ii) selecting cells which satisfies at least 2 of the marker protein expression patterns selected from the group consisting of c-Met⁺, c-Kit⁻, CD45⁻, TER119⁻ and Flk-1⁻ or a gene encoding the same.

27. The method according to claim 25, wherein the step of selecting cells further comprises selecting cells using an antibody having a specific affinity against the marker protein.

28. A method of screening for a pancreatic stem cell of a mammal, which comprises;
i) containing a population of cells with an antibody having specific affinity for a marker protein selected from the group consisting of c-Met, c-Kit, CD45 and TER119, or a gene encoding the same; and
ii) fractionating the population of cells to obtain a cell having multi-differentiation ability.

29. The method of claim 28, wherein the cells are pancreatic cells.

30. The method of claim 28, wherein the cells express a marker protein pattern of c-Met⁺, c-Kit⁻, CD45⁻ and TER119⁻.

31. The method of claim 28, wherein the cells express a marker protein pattern of c-Met⁺, c-Kit⁻, CD45⁻, TER119⁻ and Flk-1⁻.

32. An agent for the prophylaxis or treatment of a pancreatic hypofunctional disease, which comprises the pancreatic stem cell of claim 12, or a cell differentiated from the stem cell.

33. The agent of claim 32, wherein the pancreatic hypofunctional disease is a disease selected from the group consisting of diabetes, chronic pancreatitis, autoimmune pancreatitis and pancreatic functional disorder from surgical removal of all or part of a pancreas.

34. An agent for the prophylaxis or treatment of a hypofunctional disease of the liver•bile duct, which comprises the pancreatic stem cell of claim 12, or a cell differentiated from the stem cell.

35. The agent of claim 34, wherein the hypofunctional disease of the liver•bile duct is a disease selected from the group consisting of acute hepatitis, chronic hepatitis, metabolic liver disease and hepatic functional disorder from surgical removal of all or part of a liver.

36. An agent for the prophylaxis or treatment of a hypofunctional disease of the stomach•intestine, which comprises the pancreatic stem cell of claim 12, or a cell differentiated from the stem cell.

37. The agent of claim 36, wherein the hypofunctional disease of the stomach•intestine is a disease selected from the group consisting of short bowel syndrome, inflammatory bowel disease, and stomach functional disorder from surgical removal of all or part of a stomach.

38. The method of claim 2, wherein the substance having specific affinity is an antibody against the marker protein.

39. The method of claim 5, wherein the substance having specific affinity is an antibody against the marker protein.

40. A pancreatic stem cell that can be separated from the pancreas of a mammal by the method described in claim 2.

41. A pancreatic stem cell that can be separated from the pancreas of a mammal by the method described in claim 7.

42. A pancreatic stem cell that can be separated from the pancreas of a mammal by the method described claim 8.

43. The pancreatic stem cell of claim 40, which shows 4 markers of c-Met, c-Kit, CD45 and TER119 in a pattern of c-Met⁺, c-Kit⁻, CD45⁻, TER119⁻.

44. The pancreatic stem cell of claim 41, which shows 4 markers of c-Met, c-Kit, CD45 and TER119 in a pattern of c-Met⁺, c-Kit⁻, CD45⁻, TER119⁻.

45. The pancreatic stem cell of claim 42, which shows 4 markers of c-Met, c-Kit, CD45 and TER119 in a pattern of c-Met⁺, c-Kit⁻, CD45⁻, TER119⁻.

46. The pancreatic stem cell of claim 40, which shows 5 markers of c-Met, c-Kit, CD45, TER119 and Flk-1 in a pattern of c-Met⁺, c-Kit⁻, CD45⁻, TER119⁻, Flk-1⁻.

47. The pancreatic stem cell of claim 41, which shows 5 markers of c-Met, c-Kit, CD45, TER119 and Flk-1 in a pattern of c-Met⁺, c-Kit⁻, CD45⁻, TER119⁻, Flk-1⁻.

48. The pancreatic stem cell of claim 42, which shows 5 markers of c-Met, c-Kit, CD45, TER119 and Flk-1 in a pattern of c-Met⁺, c-Kit⁻, CD45⁻, TER119⁻, Flk-1⁻.

49. A method of screening a substance that induces differentiation into liver•bile duct or stomach•intestine of a mammal, which comprises the following steps:
(i) a step of reacting a pancreatic stem cell of claim 13 with a test substance, and
(ii) a step of determining the expression of a liver•bile duct or stomach•intestine marker in the cell after the reaction.

50. A method of screening a substance that regulates the function of liver•bile duct or stomach•intestine of a mammal, which comprises the following steps:
(i) a step of reacting a pancreatic stem cell of claim 13 or a cell differentiated from the stem cell with a test substance, and
(ii) a step of determining the expression of a liver•bile duct or stomach•intestine marker in the cell after the reaction.

51. A pharmaceutical composition, which comprises:
i) a cloned pluripotent pancreatic stem cell of claim 19; and
ii) a pharmaceutically acceptable carrier.

52. A purified composition, which comprises: a cloned pluripotent pancreatic stem cell of claim 19.

53. Tissue regenerated from a cloned pluripotent pancreatic stem cell of claim 19.

54. An organ regenerated from a cloned pluripotent pancreatic stem cell of claim 19.

55. A method of transplanting a cloned pluripotent pancreatic stem cell into a host, which comprises:
i) obtaining the cloned pluripotent pancreatic stem cell of claim 19; and
ii) transplanting said stem cell into the host.

56. The method according to claim 26, wherein the step of selecting cells further comprises selecting cells using an antibody having a specific affinity against the marker protein.

57. An agent for the prophylaxis or treatment of a pancreatic hypofunctional disease, which comprises the pancreatic stem cell of claim 13, or a cell differentiated from the stem cell.

58. An agent for the prophylaxis or treatment of a pancreatic hypofunctional disease, which comprises the pancreatic stem cell of claim 18, or a cell differentiated from the stem cell.

59. An agent for the prophylaxis or treatment of a pancreatic hypofunctional disease, which comprises the pancreatic stem cell of claim 19, or a cell differentiated from the stem cell.

60. The agent of claim 57, wherein the pancreatic hypofunctional disease is a disease selected from the group consisting of diabetes, chronic pancreatitis, autoimmune pancreatitis and pancreatic functional disorder from surgical removal of all or part of a pancreas.

61. The agent of claim 58, wherein the pancreatic hypofunctional disease is a disease selected from the group consisting of diabetes, chronic pancreatitis, autoimmune pancreatitis and pancreatic functional disorder from surgical removal of all or part of a pancreas.

62. The agent of claim 59, wherein the pancreatic hypofunctional disease is a disease selected from the group consisting of diabetes, chronic pancreatitis, autoimmune pancreatitis and pancreatic functional disorder from surgical removal of all or part of a pancreas.

63. An agent for the prophylaxis or treatment of a hypofunctional disease of the liver•bile duct, which comprises the pancreatic stem cell of claim 13, or a cell differentiated from the stem cell.

64. An agent for the prophylaxis or treatment of a hypofunctional disease of the liver•bile duct, which comprises the pancreatic stem cell of claim 18, or a cell differentiated from the stem cell.

65. An agent for the prophylaxis or treatment of a hypofunctional disease of the liver•bile duct, which comprises the pancreatic stem cell of claim 19, or a cell differentiated from the stem cell.

66. The agent of claim 63, wherein the hypofunctional disease of the liver•bile duct is a disease selected from the group consisting of acute hepatitis, chronic hepatitis, metabolic liver disease and hepatic functional disorder from surgical removal of all or part of a liver.

67. The agent of claim 64, wherein the hypofunctional disease of the liver•bile duct is a disease selected from the group consisting of acute hepatitis, chronic hepatitis, metabolic liver disease and hepatic functional disorder from surgical removal of all or part of a liver.

68. The agent of claim 65, wherein the hypofunctional disease of the liver•bile duct is a disease selected from the group consisting of acute hepatitis, chronic hepatitis, metabolic liver disease and hepatic functional disorder from surgical removal of all or part of a liver.

69. An agent for the prophylaxis or treatment of a hypofunctional disease of the stomach•intestine, which comprises the pancreatic stem cell of claim 13, or a cell differentiated from the stem cell.

70. An agent for the prophylaxis or treatment of a hypofunctional disease of the stomach•intestine, which comprises the pancreatic stem cell of claim 18, or a cell differentiated from the stem cell.

71. An agent for the prophylaxis or treatment of a hypofunctional disease of the stomach•intestine, which comprises the pancreatic stem cell of claim 19, or a cell differentiated from the stem cell.

72. The agent of claim 69, wherein the hypofunctional disease of the stomach•intestine is a disease selected from the group consisting of short bowel syndrome, inflammatory bowel disease, and stomach functional disorder from surgical removal of all or part of a stomach.

73. The agent of claim 70, wherein the hypofunctional disease of the stomach•intestine is a disease selected from the group consisting of short bowel syndrome, inflammatory bowel disease, and stomach functional disorder from surgical removal of all or part of a stomach.

74. The agent of claim 71, wherein the hypofunctional disease of the stomach•intestine is a disease selected from the group consisting of short bowel syndrome, inflammatory bowel disease, and stomach functional disorder from surgical removal of all or part of a stomach.
